(19) United States
(12) Patent Application Publication
(10) Pub. No.: US 2021/0093739 A1
(43) Pub. Date: Apr. 1, 2021

(54) DISINFECTION SYSTEM AND METHODS USING NITRIC ACID VAPOR

(71) Applicant: 3M INNOVATIVE PROPERTIES COMPANY, St. Paul, MN (US)

(72) Inventors: Caleb T. Nelson, Woodbury, MN (US); Drew M. Williamson, Maplewood, MN (US); Assumpta A.G. Bennaars-Eiden, Andover, MN (US)

(21) Appl. No.: 15/733,243

(22) PCT Filed: Dec. 26, 2018
(86) PCT No.: PCT/IB2018/060623

§ 371 Date: Jun. 16, 2020

Related U.S. Application Data
(60) Provisional application No. 62/611,648, filed on Dec. 29, 2017.

(51) Int. Cl. A61L 2/20; A61L 2/26; A61B 1/12

(52) U.S. Cl. CPC A61L 2/20; A61L 2101/20; A61B 1/125; A61L 2/26

(57) ABSTRACT

A disinfection system including an enclosed chamber and a source of a disinfecting vapor connected to the enclosed chamber, wherein the disinfecting vapor includes nitric acid. Methods of disinfecting a contaminated article are also described, the methods including placing the contaminated article within an enclosed chamber of a disinfection system and exposing the contaminated article within the enclosed chamber to a source of a disinfecting vapor including nitric acid for an exposure time sufficient to disinfect the contaminated article by achieving a reduction in colony forming units of the disinfected contaminated article relative to the contaminated article. Exposing the contaminated article within the enclosed chamber to the source of the disinfecting vapor may include alternately exposing the contaminated article to the disinfecting vapor for a first time interval, and exposing the contaminated article to a water vapor for a second time interval.

### FIELD

The present disclosure relates generally to the disinfection or sterilization of medical apparatus and articles, and more particularly to the application of nitric acid vapor to effect disinfection or sterilization of medical articles such as medical instruments or endoscope lumens.

### BACKGROUND

A reliable supply of sterile apparatus, instruments and supplies is vitally important to modern medical practice. Various types of apparatus are known for disinfecting or sterilizing reusable goods within a hospital setting including, for example, steam autoclaves. U.S. Pat. No. 4,301,113 (Alguire et al); U.S. Pat. No. 4,294,804 (Baran); U.S. Pat. No. 5,317,896 (Sheth et al); U.S. Pat. No. 5,399,314 (Samuel et al); U.S. Pat. No. 3,571,563 (Shulz); U.S. Pat. No. 3,054,270 (Huston); and U.S. Pat. No. 3,564,861 (Andersen et al), discuss sterilization apparatus and their control systems.

Goods which cannot withstand autoclaving temperatures can be sterilized with sterilizers using a biocidal gas such as ethylene oxide. Although ethylene oxide, nitrogen dioxide and other gaseous nitrogen oxides have been used as disinfection or sterilizing agents, these gases also exhibit undesirable toxicity and, in the case of ethylene oxide, flammability. For at least these reasons, the art has sought alternative disinfection or sterilizing agents.

### SUMMARY

The present disclosure provides a disinfection or sterilization system and disinfection or sterilization methods employing a disinfecting vapor containing nitric acid vapor as a disinfection or sterilizing agent.

Thus, in one aspect, the present disclosure describes a disinfection system including an enclosed chamber, and a source of a disinfecting vapor connected to the enclosed chamber, wherein the disinfecting vapor includes nitric acid. Optionally, the enclosed chamber is connected to a vacuum pump.

In another aspect, the present disclosure describes a method of disinfecting a contaminated article, including placing the contaminated article within an enclosed chamber of a disinfection system, and exposing the contaminated article within the enclosed chamber to a source of a disinfecting vapor including nitric acid for an exposure time sufficient to disinfect the contaminated article by achieving a reduction in colony forming units of the disinfected contaminated article relative to the contaminated article. Optionally, the enclosed chamber is connected to a vacuum pump.

Additional exemplary embodiments within the scope of the present disclosure are provided in the following Listing of Exemplary Embodiments.

### LISTING OF EXEMPLARY EMBODIMENTS

A. A disinfection system including an enclosed chamber, and a source of a disinfecting vapor connected to the enclosed chamber, wherein the disinfecting vapor includes nitric acid, optionally wherein the enclosed chamber is connected to a vacuum pump.
B. The disinfection system of Embodiment A, wherein the disinfecting vapor further includes a gas selected from the group consisting of molecular oxygen, molecular nitrogen, helium, neon, argon, krypton, or a combination thereof.
C. The disinfection system of Embodiment B, wherein the disinfecting vapor includes air.
D. The disinfection system of any preceding Embodiment, further including a source of a water vapor in a gas connected to the enclosed chamber, wherein the gas is selected from the group consisting of molecular oxygen, molecular nitrogen, helium, neon, argon, krypton, or a combination thereof optionally wherein a relative humidity of the water vapor in the gas is at least 20%.
E. The disinfection system of any preceding Embodiment, further including a device for removing at least a portion of the nitric acid from the disinfecting vapor, optionally wherein the device includes a material selected from the group consisting of a basic-functional compound, a reducing agent, a basic absorbent, a basic adsorbent, a catalyst, activated carbon, a molecular sieve, or a combination thereof.
F. The disinfection system of any preceding Embodiment, further including a contaminated article undergoing disinfection, optionally wherein the contaminated article is contaminated with at least one of a bio-film comprised of a plurality of microorganisms, a plurality of microorganisms, a bio-film comprised of a plurality of microbial spores, a plurality of microbial spores, a bio-film comprised of a plurality of fungal spores, or a plurality of fungal spores.
G. The disinfection system of Embodiment F, wherein the contaminated article is a medical article, optionally wherein the medical article is selected from the group consisting of a medical dressing, a medical instrument, a medical device, or a combination thereof.
H. The disinfection system of Embodiment G, wherein the medical device is an endoscope having a hollow lumen, further wherein the disinfecting vapor is passed through the hollow lumen of the endoscope.
I. The disinfection system of any one of Embodiments F-H, wherein the bio-film includes a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Shigella flexiniri, and a combination thereof.
J. A method of disinfecting a contaminated article, including placing the contaminated article within an enclosed chamber of a disinfection system, optionally wherein the enclosed chamber is connected to a vacuum pump; and exposing the contaminated article within the enclosed chamber to a source of a disinfecting vapor including nitric acid for an exposure time sufficient to disinfect the contaminated article by achieving a reduction in colony forming units of the disinfected contaminated article relative to the contaminated article, optionally wherein the exposure time is at most ten minutes.
K. The method of Embodiment J, wherein the disinfecting vapor further includes a gas selected from the group consisting of molecular oxygen, molecular nitrogen, helium, neon, argon, krypton, or a combination thereof.
L. The method of claim Embodiment K, wherein the disinfecting vapor comprises air.
M. The method of any one of Embodiments J, K or L, wherein the exposing of the contaminated article within the enclosed chamber to the source of the disinfecting vapor includes alternately exposing the contaminated article to the disinfecting vapor for a first time interval, and exposing the contaminated article to a water vapor for a second time interval, optionally wherein the alternately exposing the contaminated article to the disinfecting vapor for the first time interval, and exposing the contaminated article to the water vapor for the second time interval, is carried out at least two times.
N. The method of any one of Embodiments J, K, L or M, further including removing the nitric acid from the disinfecting vapor using a material selected from the group consisting of a basic-functional compound, a reducing agent, a basic absorbent, a basic adsorbent, a catalyst, activated carbon, a molecular sieve, or a combination thereof
O. The method of any one of Embodiments J, K, L, M or N, wherein the contaminated article is contaminated with at least one of a bio-film including a plurality of microorganisms, a plurality of microorganisms, a bio-film including a plurality of microbial spores, a plurality of microbial spores, a bio-film including a plurality of fungal spores, or a plurality of fungal spores.
P. The method of any one of Embodiments J, K, L, M, N or O, wherein the contaminated article is a medical article, optionally wherein the medical article is selected from a medical dressing, a medical instrument, a medical device, or a combination thereof.
Q. The method of Embodiment P, wherein the medical device is an endoscope having a hollow lumen, further wherein the disinfecting vapor is passed through the hollow lumen of the endoscope.
R. The method of any one of Embodiments O, P or Q, wherein the contaminated article is contaminated with a bio-film including a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Shigella flexiniri, and a combination thereof, further wherein the exposure time is at least 1 minute and the reduction in colony forming units of the disinfected article relative to the contaminated article is at least a 2-log₁₀ and up to an 11-log₁₀ reduction, optionally wherein the exposure time is at most six minutes.
S. The method of any one of Embodiments O, P, Q or R, wherein the contaminated article is contaminated with a plurality of microorganisms, further wherein the exposure time is at least 1 minute and the reduction in colony forming units of the disinfected article relative to the contaminated article is at least 4-log₁₀ and up to 9-log₁₀, optionally wherein the exposure time is at most six minutes.
T. The method of any one of any one of Embodiments O, P, Q, R or S, wherein the contaminated article is contaminated with a plurality of microbial spores or a plurality of fungal spores, wherein the exposure time is at least 1 minute and the reduction in colony forming units of the disinfected article relative to the contaminated article is at least 6-log₁₀ and up to 10-log₁₀, optionally wherein the exposure time is at most six minutes.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying figures, in which:

FIG. 1 is a schematic view of an exemplary disinfection or sterilization system of one embodiment of the present disclosure.

FIG. 2 is a schematic view of an exemplary disinfection or sterilization system of another embodiment of the present disclosure.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

Certain terms are used throughout the description and the claims that, while for the most part are well known, may require some explanation. It should understood that, as used herein unless a different definition is expressly provided in the claims or elsewhere in the specification, including the drawings, the following terms have the meaning defined in the following Glossary.

### Glossary

The terms “disinfection” and “sterilization” and their derivative forms are used to describe systems and methods for achieving a reduction in the quantity of infectious agents such as bacteria, viruses, spores, and other microorganisms, on a surface of an article such as a medical article (e.g., a medical instrument or endoscope lumen). It will be understood that generally, sterilization is disinfection that achieves a higher reduction in the quantity of infectious agents. Throughout the specification, including the claims, the term “disinfection” subsumes the term “sterilization.”

The terms “about” or “approximately” with reference to a numerical value or a shape means+/−five percent of the numerical value or property or characteristic, but expressly includes the exact numerical value. For example, a viscosity of “about” 1 Pa-sec refers to a viscosity from 0.95 to 1.05 Pa-sec, but also expressly includes a viscosity of exactly 1 Pa-sec.

As used in this specification and the appended embodiments, the singular forms “a”, “an”, and “the” include plural referents unless the content clearly dictates otherwise. Thus, for example, reference to fine fibers containing “a compound” includes a mixture of two or more compounds. As used in this specification and the appended embodiments, the term “or” is generally employed in its sense including “and/or” unless the content clearly dictates otherwise.

The term “substantially” with particular reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

As used in this specification, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

Various exemplary embodiments of the disclosure will now be described with particular reference to the Drawings.

### Exemplary Disinfection or Sterilization Systems

The present disclosure describes disinfection or sterilization systems including an enclosed chamber, and a source of a disinfecting vapor connected to the enclosed chamber, wherein the disinfecting vapor includes nitric acid. The disinfection or sterilization systems may be further described with respect to two distinct embodiments.

### Embodiment 1: Atmospheric Pressure Vaporized Nitric Acid Disinfection or Sterilization Process

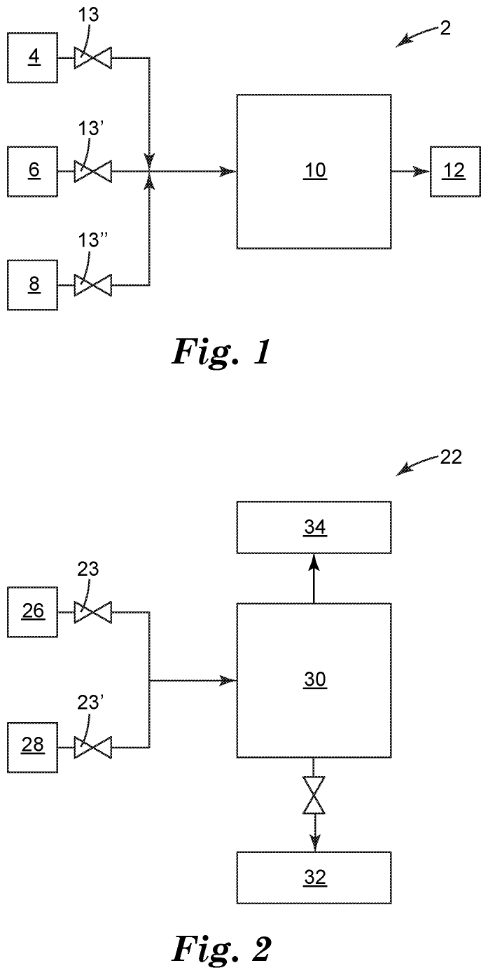

Turning to FIG. 1, a schematic view of an exemplary disinfection system 2 of one embodiment of the present disclosure is shown. The disinfection system 2 includes an enclosed disinfection chamber 10 in flow communication with a source of disinfecting vapor 6 including nitric acid. An optional flow controller 13′ (e.g., an electronic mass flow controller or a flow control valve) may be used to regulate the flow of the disinfecting vapor 6 into the disinfection chamber 10. An article (not shown in FIG. 1) to be disinfected or sterilized may be placed with the disinfection chamber 10, for example through a sealable door, window, or port (not shown in FIG. 1), and the chamber 10 may be thereafter sealed to achieve disinfection or sterilization.

Optionally, the disinfection system 2 further includes a source of a gas 4 in flow communication with the enclosed disinfection chamber 10. An optional flow controller 13 (e.g., an electronic mass flow controller or a flow control valve) may be used to regulate the flow of the gas 4 into the disinfection chamber 10. The flow of the source of gas 4 may be combined with the flow of the disinfecting vapor 6 into the disinfection chamber 10 after flow controller 13′ as shown in FIG. 1, or alternatively, may be combined with the disinfecting vapor 6 before flow controller 13′ (not shown), or may even flow directly into the disinfection chamber 10 (not shown).

The source of gas 4 may include molecular oxygen, molecular nitrogen, helium, neon, argon, krypton, or a combination thereof; air may be advantageously selected. The disinfecting vapor 6 may, in certain exemplary embodiments, include molecular oxygen, molecular nitrogen, helium, neon, argon, krypton, or a combination thereof. The source of gas 4 may be air or a specific blend including molecular oxygen and nitrogen at a specified ratio, and may be pressurized or unpressurized, as convenient. If from an unpressurized source, a compressor may be used to pressurize the gas to a convenient pressure.

Optionally, the disinfection system 2 further includes a source of water vapor 8 in a gas connected to the enclosed chamber 10. An optional flow controller 13″ (e.g., an electronic mass flow controller or a flow control valve) may be used to regulate the flow of the source of water vapor 8 in a gas into the disinfection chamber 10. The flow of the source of water vapor 8 in a gas may be combined with the flow of the disinfecting vapor 4 into the disinfection chamber 10 after flow controller 13 as shown in FIG. 1, or alternatively, may be combined with the disinfecting vapor 4 before flow controller 13 (not shown), or may even flow directly into the disinfection chamber 10 (not shown).

The gas may include molecular oxygen, molecular nitrogen, helium, neon, argon, krypton, or a combination thereof; air may be advantageously selected. The relative humidity of the source of water vapor 8 in a gas may, in some embodiments, be at least 20%, 25%, 30%, 40%, 50%, 60% or even at least 70%, and preferably is less than 100%, 95%, 90%, 80%, or even 75%, depending on the temperature within the disinfection chamber 10.

The temperature within the disinfection chamber 10 may, in some embodiments, be advantageously maintained at a temperature to avoid condensation of liquid water within the disinfection chamber 10. Typically, the temperature within the chamber is maintained from 20° C. to 100° C., 21° C. to 95° C., or even 22° C. to 90° C., although temperatures greater than 100° C. may be advantageously used. Generally, the temperature within the disinfection chamber 10 should be maintained below the temperature at which the contaminated article would be damaged or degraded.

Various devices for vaporizing or atomizing the nitric acid in the disinfecting vapor, or for adding water vapor to the gas, such as water-containing or nitric acid-containing gas bubblers, spargers, atomizers, and wick-type humidifiers (not shown in FIG. 1), may all be advantageously used. These devices may be inserted anywhere from the disinfection chamber 10 up to and including the source of disinfecting vapor 6, the source of gas 4, and/or the source of water vapor 8.

The optional flow controllers 13, 13′ and 13″ may independently be selected as any device for regulating the flowrate of the disinfecting vapor 6. Suitable devices include pressure regulators, flow control valves, ball-in-tube flowmeters (rotameters), electronic mass flow controllers, or other similar devices.

Optionally, the disinfection chamber 10 is connected to a device 12 for removing at least a portion of the nitric acid from the disinfecting vapor 6. The device 12 for removing at least a portion of the nitric acid from the disinfecting vapor 6 may include a material selected from a basic-functional compound, a reducing agent, a basic absorbent, a basic adsorbent, a catalyst, activated carbon, a molecular sieve, or a combination thereof.

In convenient embodiments, the device 12 for removing at least a portion of the nitric acid from the disinfecting vapor 6 is a filter including an alkaline element such as sodium bicarbonate to neutralize any remaining acidic species. An optional filter element such as activated carbon to remove oxidizing species such as ozone is also conveniently present.

It is generally preferred that 100%, or alternatively at least 99%, 98%, 97%, 96%, 95%, 94%, 93%, 92%, 91%, or even at least 90% of the nitric acid in the disinfecting vapor 6 be removed by the device 12 for removing at least a portion of the nitric acid from the disinfecting vapor 6. After removing the desired portion of the nitric acid from the disinfecting vapor 6, the remaining disinfecting vapor 6 can be released to ambient conditions.

### Embodiment 2: Vacuum Pulsed Vaporized Nitric Acid Disinfection or Sterilization Process

Turning to FIG. 2, a schematic view of an exemplary disinfection system 22 of an alternative embodiment of the present disclosure is shown. The disinfection system 22 includes an enclosed disinfection chamber 30 in flow communication with a source of disinfecting vapor 26 including nitric acid. An optional flow controller 23 may be used to regulate the flow of the disinfecting vapor 26 into the disinfection chamber 30. An article (not shown in FIG. 2) to be disinfected or sterilized may be placed with the disinfection chamber 30, for example through a sealable door, window, or port (not shown in FIG. 2), and the chamber 30 may be thereafter sealed to achieve disinfection or sterilization.

The enclosed chamber 30 is connected to a vacuum pump 32 (e.g., a single or multi-stage rotary vacuum pump, a molecular jet pump, a diffusion pump, or the like) which may be isolated from the disinfection chamber 30 by an optional valve 36. A vacuum gauge 34 (e.g., a pressure gauge, manometer, hot-filament ionization gauge, McLeod gauge, Penning gauge, Pirani gauge or the like) may be connected to the enclosed chamber 30 to permit monitoring of the pressure within the chamber 30.

The disinfection system 22 includes a source of water vapor 28 in a gas connected to the disinfection chamber 30. An optional flow controller 23′ (e.g., an electronic mass flow controller or a flow control valve) may be used to regulate the flow of the source of water vapor 28 in a gas into the disinfection chamber 10. The flow of the source of water vapor 28 in a gas may be combined with the flow of the disinfecting vapor 26 into the disinfection chamber 30 after flow controller 23 as shown in FIG. 2, or alternatively, may be combined with the disinfecting vapor 26 before flow controller 23 (not shown), or may even flow directly into the disinfection chamber 30 (not shown).

The gas may include molecular oxygen, molecular nitrogen, helium, neon, argon, krypton, or a combination thereof; air may be advantageously selected. The relative humidity of the source of water vapor 28 in a gas may, in some embodiments, be at least 20%, 25%, 30%, 40%, 50%, 60% or even at least 70%, and preferably is less than 100%, 95%, 90%, 80%, or even 75%, depending on the temperature within the disinfection chamber 30.

The temperature within the disinfection chamber 30 may, in some embodiments, be advantageously maintained at a temperature to avoid condensation of liquid water within the disinfection chamber 30. Typically, the temperature within the chamber is maintained from 20° C. to 100° C., 21° C. to 95° C., or even 22° C. to 90° C., although temperatures greater than 100° C. may be advantageously used. Generally, the temperature within the disinfection chamber 10 should be maintained below the temperature at which the contaminated article would be damaged or degraded.

Various devices for vaporizing or atomizing the nitric acid in the disinfecting vapor, or for adding water vapor to the gas, such as water-containing or nitric acid-containing gas bubblers, spargers, atomizers, and wick-type humidifiers (not shown in FIG. 2), may all be advantageously used. These devices may be inserted anywhere from the disinfection chamber 30 up to and including the source of disinfecting vapor 26, and/or the source of water vapor 28.

The optional flow controllers 23, and 23′ may independently be selected as any device for regulating the flowrate of the disinfecting vapor 26. Suitable devices include pressure regulators, flow control valves, ball-in-tube flowmeters (rotameters), electronic mass flow controllers, or other similar devices.

Optionally, the disinfection chamber 30 is connected to a device (not shown) for removing at least a portion of the nitric acid from the disinfecting vapor 26. The device for removing at least a portion of the nitric acid from the disinfecting vapor 26 may be positioned in flow communication with the disinfection chamber 30, but is preferably placed in flow communication between the disinfection chamber 30 and the vacuum pump 32, or on the exhaust from the vacuum pump.

The device for removing at least a portion of the nitric acid from the disinfecting vapor 26 may include a material selected from a basic-functional compound, a reducing agent, a basic absorbent, a basic adsorbent, a catalyst, activated carbon, a molecular sieve, or a combination thereof. In convenient embodiments, the device for removing at least a portion of the nitric acid from the disinfecting vapor 26 is a filter including an alkaline element such as sodium bicarbonate to neutralize any remaining acidic species. An optional filter element such as activated carbon to remove oxidizing species such as ozone is also conveniently present.

It is generally preferred that 100%, or alternatively at least 99%, 98%, 97%, 96%, 95%, 94%, 93%, 92%, 91%, or even at least 90% of the nitric acid in the disinfecting vapor 26 be removed by the device for removing at least a portion of the nitric acid from the disinfecting vapor 26. After removing the desired portion of the nitric acid from the disinfecting vapor 26, the remaining disinfecting vapor 26 can be released to ambient conditions.

### Methods of Producing the Disinfecting Vapor

In each of the disinfection or sterilization systems of Embodiment 1 or Embodiment 2 as described above, a disinfecting vapor containing nitric acid is used to achieve disinfection or sterilization of a contaminated article.

As noted above, various disinfecting vapor-forming devices may be advantageously used for vaporizing the nitric acid to form the disinfecting vapor 6 or 26. In certain exemplary embodiments, the nitric acid may advantageously be vaporized from an aqueous solution by flowing a gas stream through a gas bubbler or sparger containing aqueous nitric acid, with or without heating of the aqueous nitric acid. The nitric acid may be vaporized using vacuum evaporation, atmospheric pressure evaporation, flash evaporation, or atomization into a flowing gas stream. Various vacuum evaporators, atmospheric pressure evaporators, flash evaporators, atomizers, and wick-type humidifiers may be used. These disinfecting vapor-forming devices may be inserted anywhere from the disinfection chamber 30 up to and including the source of disinfecting vapor 26, and/or the source of water vapor 28.

A number of methods may be used to produce aqueous nitric acid, which are described further below.

### Formation of Nitric Acid by Oxidation of Ammonia (Ostwald Process)

This process involves three stages: (1) oxidation of ammonia, (2) nitric oxide oxidation, and (3) the absorption of the resulting nitrogen oxides.

Stage 1:

4NH_{2(g)}+5O_{2(g)}↔4NO_{(g)}+6H₂O_{(g)}

Process Conditions:
Catalyst: Platinum (90% Pt/10% Rd is commonly used)
Pressure: 1-4 atmospheres (atm)
Temperature: ˜1100° K

Stage 2:

NO_{(g)}+O_{2(g)}↔2NO_{2(g)}↔N₂O_{4(g)}

Process Conditions:
Catalyst: None
Pressure: ˜8 atm
Temperature: ˜300 K

Stage 3:

3NO_{2(g)}+H₂O₍ₗ₎↔2HNO_{2(aq)}+NO_{(g)}

Process Conditions:
Reaction and distillation typically occur simultaneously in a staged distillation column.
Catalyst: None
Pressure: 11-14 atm
Temperature: N/A

### Formation of Nitric Acid\Via Thermal Decomposition of Copper Nitrate

This process involves two reactions carried out in a single stage.

2Cu(NO₃)₂↔2CuO₍ₛ₎+4NO_{2(g)}+O_{2(g)}

3NO_{2(g)}+H₂O₍ₗ₎↔2HNO_{2(aq)}+NO_{(g)}
Reaction and distillation to form vapor typically occur simultaneously in a staged distillation column.

Process Conditions:
Catalyst: None
Pressure: 11-14 atm
Temperature: N/A
   Nitric Acid Production by Reaction of Sodium Nitrate with Sulfuric Acid

This process involves a single stage, and may be preferred.

2NaNO₂+H₂SO_{4(aq)}↔2HNO₂+Na₂SO₄

Conditions:
The nitric acid is boiled off and condensed into a collection vessel to separate from the sodium sulfate.

### Contaminated Articles

In each of the disinfection systems of Embodiment 1 or Embodiment 2 as described above, a contaminated article undergoing disinfection is inserted into the enclosed disinfection chamber (10 or 30) and sealed. The contaminated article may be a medical article, for example, a medical article selected a medical dressing, a medical instrument, a medical device, or a combination thereof. In certain embodiments, the medical device is an endoscope having a hollow lumen, and the disinfecting vapor is passed through the hollow lumen of the endoscope to achieve disinfection or sterilization.

The contaminated article may be contaminated with at least one of a bio-film comprised of a plurality of microorganisms, a plurality of microorganisms, a bio-film comprised of a plurality of microbial spores, a plurality of microbial spores, a bio-film comprised of a plurality of fungal spores, or a plurality of fungal spores. The bio-film may include a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Shigella flexiniri, and combinations thereof.

### Exemplary Disinfection or Sterilization Processes

In another aspect, the present disclosure describes methods of disinfecting and/or sterilizing a contaminated article. The methods include placing the contaminated article within an enclosed disinfection chamber 10, 30 of a disinfection system 2, 22, and exposing the contaminated article within the enclosed disinfection chamber 10, 30 to a source of a disinfecting vapor 6, 26 including nitric acid for an exposure time sufficient to disinfect the contaminated article by achieving a reduction in colony forming units of the disinfected contaminated article relative to the contaminated article. Optionally the enclosed disinfection chamber 10, 30 is connected to a vacuum pump 32.

In exemplary embodiments, the disinfecting vapor further includes a gas selected from molecular oxygen, molecular nitrogen, helium, neon, argon, krypton, or a combination thereof. The disinfecting vapor may include air.

Exposing of the contaminated article within the enclosed chamber to the source of the disinfecting vapor may, in some embodiments, include alternately exposing the contaminated article to the disinfecting vapor for a first time interval, and exposing the contaminated article to water vapor for a second time interval. In some such embodiments, alternately exposing the contaminated article to the disinfecting vapor for the first time interval, and exposing the contaminated article to the water vapor for the second time interval, is carried out at least two times, at least three times, at least four times, at least five times, at least six times, at least seven times, at least eight times, at least nine times, or even at least 10 times. In certain such embodiments, alternately exposing the contaminated article to the disinfecting vapor for the first time interval, and exposing the contaminated article to the water vapor for the second time interval, is carried out at most 20 times, at most 10 times, at most 8 times, at most 7 times, at most 6 times, or even at most 5 times.

In certain exemplary embodiments, the method further includes removing at least a portion of the vaporized nitric acid from the gas upon achieving the desired degree of sterilization of the article. Removing the nitric acid from the disinfecting vapor may advantageously be carried out using a material selected from a basic-functional compound, a reducing agent, a basic absorbent, a basic adsorbent, a catalyst, activated carbon, a molecular sieve, or a combination thereof.

In some embodiments, removing the vaporized nitric acid from the gas may be performed with a device 12 including one or more adsorbent or absorbent materials selected from activated carbon, a chemical species with a basic functionality (e.g., an organic amine, sodium hydroxide, potassium hydroxide, lithium hydroxide, and the like), a species providing a basic adsorbent (e.g. a basic ion exchange resin), a reducing species (e.g., one or more active metals such as platinum, palladium, and the like), and a molecular sieve. In some exemplary embodiments, removing the vaporized nitric acid from the gas may be performed by directing the gas through a catalytic reducer.

In certain embodiments, the contaminated article is a medical article. The medical article may be selected from a medical dressing, a medical instrument, a medical device, or a combination thereof. In certain presently-preferred embodiments, the medical device is an endoscope having a hollow lumen, and the disinfecting vapor is passed through the hollow lumen of the endoscope.

In some exemplary embodiments, the contaminated article is contaminated with at least one of a bio-film including a plurality of microorganisms, a plurality of microorganisms, a bio-film including a plurality of microbial spores, a plurality of microbial spores, a bio-film including a plurality of fungal spores, or a plurality of fungal spores. In certain exemplary embodiments, the contaminated article is contaminated with a bio-film including a plurality of microorganisms selected from Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Shigella flexiniri, and combinations thereof.

In any of the foregoing embodiments, the exposure time is at least 1 minute, and at most 10 minutes. Preferably, the exposure time to achieve the desired level of disinfection is selected to be at most 9 minutes, 8 minutes, 7 minutes, 6 minutes, 5 minutes, or even at most 4 minutes, 3 minutes, 2 minutes, or even 1 minute.

In any of the foregoing embodiments, the reduction in colony forming units of the disinfected article relative to the contaminated article is at least a 2-log₁₀ and up to an 11-log₁₀ reduction, at least a 3-log₁₀ and up to an 11-log₁₀ reduction, at least a 4-log₁₀ and up to an 11-log₁₀ reduction, at least a 5-log₁₀ and up to an 11-log₁₀ reduction, at least a 6-log₁₀ and up to an 11-log₁₀ reduction, at least a 7-log₁₀ and up to an 11-log₁₀ reduction, at least an 8-log₁₀ and up to an 11-log₁₀ reduction, at least a 9-log₁₀ and up to an 11-log₁₀ reduction, or even at least a 10-log₁₀ and up to an 11-log₁₀ reduction.

The operation of exemplary embodiments of the present disclosure will be further described with respect to the following non-limiting detailed Examples. These examples are offered to further illustrate the various specific and preferred embodiments and techniques. It should be understood, however, that many variations and modifications may be made while remaining within the scope of the present disclosure.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

### Materials

Unless otherwise noted, all parts, percentages, ratios, etc. in the Examples and the rest of the specification are by weight. Solvents and other reagents used may be obtained from Sigma-Aldrich Chemical Company (Milwaukee, Wis.) unless otherwise noted. In addition, Table 1 provides abbreviations and a source for all materials used in the Examples below:

TABLE 1
Materials

| Materials (Part Number) | Source |
|---|---|
| Phosphate buffered saline | Sigma-Aldrich (St. Louis, MO) |
| 10X concentrate (P5493-1L) | |
| Tween ® 80 (P4780) | Sigma-Aldrich (St. Louis, MO) |
| Geobacillus | Iuvo BioScience LLC (Warsaw, NY) |
| stearothermophilus | |
| (ATCC #7953) | |
| 3M Petrifilm ® AC | 3M Company (St. Paul, MN) |
| Mini Flip Top Vial With | 3M Company (St. Paul, MN) |
| Butterfields Buffer | |
| PET film 1 mil | Mistubishi Polyester Film |
| | (Greenville, SC) |
| 68.8% Nitric Acid | Sigma-Aldrich (St. Louis, MO) |
| Steri-Lok ® 8502 | 3M Company (St. Paul, MN) |
| Aldrich vacuum trap | Sigma Aldrich (St. Louis, MO) |
| (Z549398) | |
| PTFETube (5239K11) | McMaster-Carr, Inc. (Elmhurst, IL) |
| Compressed Oxygen, Ultra | Oxygen Service Company (St. Paul, MN) |
| High Purity (UHP) | |
| Compressed Nitrogen, | Oxygen Service Company (St. Paul, MN) |
| Ultra High Purity (UHP) | |

### Preparation of Spore Samples

Samples of PET film (1×2 cm) were cut and placed in petri dishes. Then, 10 μL of Geobacillus stearothermophilus spores (˜1×10⁸ CFU/mL, vortexed 1 minute) were drop-cast onto each of the films. The films contained about 1×10⁶ spores per film. The films were allowed to dry with the petri dish lid open for at least 1 hour at ambient conditions to ensure that the samples were fully dry.

For some experiments, the films were inserted into PTFE sample tube pieces using clean tweezers with 3 films inserted per sample tube. The films were inspected to ensure there was no significant overlap of the spore spots and that the films were in the PTFE tube with the spores facing the interior of the tube. For other experiments, the films were inserted into Steri-Lok 8502 bags prior to treatment.

### Collecting Spore Samples and Colony Counting

A solution of 1× phosphate buffered saline (1×PBST) was prepared from 100 mL of phosphate buffered saline 10× concentrate, 900 mL of deionized water, and 1 g of Tween 80 surfactant. The 1×PBST solutions were mixed for 5 minutes on a stir plate and were then vacuum filtered through a 0.2 μm vacuum filter to ensure sterility and stored at 4° C. After sterilization treatments, the spore films were removed using sterile tweezers. Then the films were immediately transferred to 50 mL tubes containing 25 mL of 1×PBST to neutralize the pH and all oxidizing species. The 50 mL tubes were immediately vortex-mixed, then sonicated for 20 min and vortex-mixed again to ensure all of the spores were removed from the surface.

One milliliter samples of the buffer solutions containing spores were diluted in Butterfield's buffer. Serial dilutions with 10-, 100-, and 1000-fold reductions in concentration were carried out because the original samples contained 10⁶ colonies and it was necessary to reduce the concentration for counting. Then one milliliter of the appropriate dilution series made from exposed film samples and from the unexposed (control) samples in 1×PBST were spread onto Petrifilm®. The Petrifilm® plates were placed onto an aluminum tray and the spores were put in an oven at an optimum growth temperature of 56° C. so that the colonies could grow if colony forming units (CFU) were present.

After the spores were incubated, the colony forming units were counted. In each case, the control samples of untreated spore films were used as the standard. For ideal quantification of kill, the number of CFU per Petrifilm® was quantified in the range of 20-200. Based on the number of CFU's and the known dilution concentration, it was possible to calculate the number of original CFU's from the controls or treated spore films and quantify spore kill.

### Apparatus and Method

The sterilization apparatus used in these examples is shown schematically in FIG. 2, and includes an enclosed disinfection chamber 30, a vacuum pump 32, a vacuum gauge 34, and solenoid valves 23 and 23′ connecting sources of nitric acid and water. The disinfection chamber 30 had a volume of 10 L.

### Example 1

In separate vessels, 10 mL of 68.8% nitric acid and 10 mL of water were dosed. Kill efficacy was tested by first preparing G. stearothermophilus samples according to the test method above. Three strips of inoculated PET were placed inside a 3M Steri-Lok 8502 bag. The bag was then heat sealed and placed in the chamber of the sterilization apparatus. The chamber was pumped down to a base pressure of 0.5 torr. The valve to the roughing pump was then closed, isolating the vacuum chamber. The valve to the nitric acid solution was then opened for a time specified in Table 2. Subsequently, the valve to the nitric acid delivery system was closed, and the valve to the water source was opened for the time specified in Table 2. The valve to the water source was then closed, and the valve to the pump was opened, bringing the chamber back to base pressure.

TABLE 2
Kill Efficacy as a Function of Exposure
Time and Number of Cycles.

| HNO₃ | H₂O | | |
|---|---|---|---|
| Exposure | Exposure | | |
| Time | Time | Number of | |
| (seconds) | (seconds) | Cycles | Results |
| 0 | 0 | 0 | +++ |
| 60 | 60 | 2 | − |
| 60 | 60 | 1 | − |
| 30 | 30 | 1 | − |
| 15 | 15 | 1 | +++ |
| 60 | 0 | 2 | +− |

The process was repeated for the indicated number of cycles. After completion of the cycles, the valve to the pump was closed and humidified air was used to vent the system up to 700 Torr. The chamber was then evacuated to the base pressure. This process was repeated three times before allowing the chamber to equilibrate at atmospheric pressure. After exposure, the chamber was vented, sample films were collected, and spores were counted as described above. The first line of results in Table 2 represents the control condition. The control samples had an average of 6.1 Log₁₀ CFU/sample. Subsequent samples were counted with no dilution. Samples with no growth are shown as negative results, and those with colony forming units are shown as positive results.

### Example 2

### Sterilization Efficacy Inside a Lumen

Kill efficacy inside a narrow lumen was tested by first preparing G. stearothermophilus samples according to the test method above. Three strips of inoculated PET were placed in the middle of a 6-foot length of PTFE tubing having ⅛″ diameter lumen. The tube was subsequently placed in the vacuum chamber described in Example 1. The method described in Example 1 was used with 60 second exposures of HNO₃ and H₂O with 2 cycles. After exposure, the chamber was vented, the sample films were collected, and spores were counted as described above. All treated samples showed no growth after 48 hours.

### Example 3

### Atmospheric Vaporization of Nitric Acid

A sterilization apparatus was constructed using a 25 mm OD×200 mm Sigma-Aldrich vacuum trap as a bubbling unit. An aqueous solution of HNO₃ in H₂O was prepared at 45% HNO3 by weight. 5.4 mL of the solution was injected in the bottom of the bubbling unit. A mixture of 500 standard cubic centimeters per minute (SCCM) of oxygen and 1750 SCCM of nitrogen were blown through the bubbling unit. The output from the bubbling unit was transported through PTFE tubing to a tee where it was mixed with an additional 750 SCCM of nitrogen. The total mixture was transported from the tee through ⅛″ ID PTFE tubing. At the 6 feet (about 1.83 m) downstream from the vapor mixing apparatus, spore coated films were inserted into the PTFE tubing. These films were exposed to the gaseous mixture for a total of 5 minutes. The initial spore concentration was measured to be 6.2 Log₁₀ CFU/Sample. After exposure to the vaporized HNO₃ mixture, no colony forming units were observed.

### Comparative Example 1

### Liquid HNO₃

A liquid solution of HNO₃ and H₂O was prepared at 5% HNO3 by weight. G. stearothermophilus coated films were prepared. Thirty microliters of the HNO₃ solution was drop cast onto the spores, covering the inoculated area. The solution was allowed to dwell on the film for 5 minutes before the film was buffered in 25 mL of 1×PBST. Subsequent recovery was completed as previously described. Prior to the HNO₃ exposure, 5.9 Log₁₀ CFU/sample were recovered. After 5 minutes exposure to the HNO₃ solution, 5.4 Log₁₀ CFU/sample were recovered.

Reference throughout this specification to “one embodiment,” “certain embodiments,” “one or more embodiments” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus the appearances of the phrases such as “in one or more embodiments,” “in certain embodiments,” “in one embodiment” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

Furthermore, all publications and patents referenced herein are incorporated by reference in their entirety to the same extent as if each individual publication or patent was specifically and individually indicated to be incorporated by reference. Various exemplary embodiments have been described.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove. These and other embodiments are within the scope of the following claims.

1. A disinfection system, comprising:
an enclosed chamber; and
a source of a disinfecting vapor connected to the enclosed chamber, wherein the disinfecting vapor comprises nitric acid, optionally wherein the enclosed chamber is connected to a vacuum pump.

2. The disinfection system of claim 1, wherein the disinfecting vapor further comprises a gas selected from the group consisting of molecular oxygen, molecular nitrogen, helium, neon, argon, krypton, or a combination thereof.

3. The disinfection system of claim 2, wherein the disinfecting vapor comprises air.

4. The disinfection system of claim 1, further comprising a source of a water vapor in a gas connected to the enclosed chamber, wherein the gas is selected from the group consisting of molecular oxygen, molecular nitrogen, helium, neon, argon, krypton, or a combination thereof, optionally wherein a relative humidity of the water vapor in the gas is at least 20%.

5. The disinfection system of claim 1, further comprising a device for removing at least a portion of the nitric acid from the disinfecting vapor, optionally wherein the device comprises a material selected from the group consisting of a basic-functional compound, a reducing agent, a basic absorbent, a basic adsorbent, a catalyst, activated carbon, a molecular sieve, or a combination thereof.

6. The disinfection system of claim 1, further comprising a contaminated article undergoing disinfection, optionally wherein the contaminated article is contaminated with at least one of a bio-film comprised of a plurality of microorganisms, a plurality of microorganisms, a bio-film comprised of a plurality of microbial spores, a plurality of microbial spores, a bio-film comprised of a plurality of fungal spores, or a plurality of fungal spores.

7. The disinfection system of claim 6, wherein the contaminated article is a medical article, optionally wherein the medical article is selected from the group consisting of a medical dressing, a medical instrument, a medical device, or a combination thereof.

8. The disinfection system of claim 7, wherein the medical device is an endoscope comprised of a hollow lumen, further wherein the disinfecting vapor is passed through the hollow lumen of the endoscope.

9. The disinfection system of claim 6, wherein the bio-film is comprised of a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Shigella flexiniri, and a combination thereof.

10. A method of disinfecting a contaminated article, comprising:
placing the contaminated article within an enclosed chamber of a disinfection system, optionally wherein the enclosed chamber is connected to a vacuum pump; and
exposing the contaminated article within the enclosed chamber to a source of a disinfecting vapor comprising nitric acid for an exposure time sufficient to disinfect the contaminated article by achieving a reduction in colony forming units of the disinfected contaminated article relative to the contaminated article, optionally wherein the exposure time is at most ten minutes.

11. The method of claim 10, wherein the disinfecting vapor further comprises a gas selected from the group consisting of molecular oxygen, molecular nitrogen, helium, neon, argon, krypton, or a combination thereof.

12. The method of claim 11, wherein the disinfecting vapor comprises air.

13. The method of claim 11, wherein the exposing of the contaminated article within the enclosed chamber to the source of the disinfecting vapor comprises alternately exposing the contaminated article to the disinfecting vapor for a first time interval, and exposing the contaminated article to a water vapor for a second time interval, optionally wherein the alternately exposing the contaminated article to the disinfecting vapor for the first time interval, and exposing the contaminated article to the water vapor for the second time interval, is carried out at least two times.

14. The method of claim 10, further comprising removing the nitric acid from the disinfecting vapor using a material selected from the group consisting of a basic-functional compound, a reducing agent, a basic absorbent, a basic adsorbent, a catalyst, activated carbon, a molecular sieve, or a combination thereof.

15. The method of claim 10, wherein the contaminated article is contaminated with at least one of a bio-film comprised of a plurality of microorganisms, a plurality of microorganisms, a bio-film comprised of a plurality of microbial spores, a plurality of microbial spores, a bio-film comprised of a plurality of fungal spores, or a plurality of fungal spores.

16. The method of claim 15, wherein the contaminated article is a medical article, optionally wherein the medical article is selected from a medical dressing, a medical instrument, a medical device, or a combination thereof.

17. The method of claim 16, wherein the medical device is an endoscope comprised of a hollow lumen, further wherein the disinfecting vapor is passed through the hollow lumen of the endoscope.

18. The method of claim 15, wherein the contaminated article is contaminated with a bio-film comprising a plurality of microorganisms selected from the group consisting of Geobacillus stearothermophilus, Bacillus subtilis, Bacillus atrophaeus, Bacillus megaterium, Bacillus coagulans, Clostridium sporogenes, Bacillus pumilus, Aspergillus brasiliensis, Aspergillus oryzae, Aspergillus niger, Aspergillus nidulans, Aspergillus flavus, Clostridium difficile, Mycobacterium terrae, Mycobacterium tuberculosis, Mycobacterium bovis, Escherichia coli, Staphylococcus aureus, Pseudomonas aeruginosa, Staphylococcus epidermidis, Staphyolococcus lugdunensis, Staphylococcus saprophyticus, Enterococcus faecium, Enterococcus faecalis, Propionobacterium acnes, Klebsiella pneumoniae, Enterobacter cloacae, Proteus mirabilus, Salmonella enterica, Salmonella typhi, Shigella flexiniri, and a combination thereof, further wherein the exposure time is at least 1 minute and the reduction in colony forming units of the disinfected article relative to the contaminated article is at least a 2-log₁₀ and up to an 11-log₁₀ reduction, optionally wherein the exposure time is at most six minutes.

19. The method of claim 15, wherein the contaminated article is contaminated with a plurality of microorganisms, further wherein the exposure time is at least 1 minute and the reduction in colony forming units of the disinfected article relative to the contaminated article is at least 4-log₁₀ and up to 9-log₁₀, optionally wherein the exposure time is at most six minutes.

20. The method of any one of claim 15, wherein the contaminated article is contaminated with a plurality of microbial spores or a plurality of fungal spores, wherein the exposure time is at least 1 minute and the reduction in colony forming units of the disinfected article relative to the contaminated article is at least 6-log₁₀ and up to 10-log₁₀, optionally wherein the exposure time is at most six minutes.
